(19) United States
(12) Patent Application Publication
(10) Pub. No.: US 2022/0372446 A1
(43) Pub. Date: Nov. 24, 2022

(54) HIGH-THROUGHPUT CULTURE OF IPSC-DERIVED ALVEOLAR CELLS

(71) Applicant: The General Hospital Corporation, Boston, MA (US)

(72) Inventors: Harald C. Ott, Wenham, MA (US); Sydney Jeffs, Boston, MA (US)

(21) Appl. No.: 17/772,251

(22) PCT Filed: Oct. 30, 2020
(86) PCT No.: PCT/US2020/058350

§ 371 Date: Apr. 27, 2022

Related U.S. Application Data
(60) Provisional application No. 62/945,834, filed on Dec. 9, 2019.
(60) Provisional application No. 62/927,797, filed on Oct. 30, 2019.

(51) Int. Cl. C12N 5/071; C12M 1/12; C12M 3/00

(52) U.S. Cl. CPC C12N 5/0688; C12M 25/14; C12M 21/08; C12N 2506/45; C12N 2533/90; C12N 2513/00; C12N 2501/117; C12N 2501/119

(57) ABSTRACT

Provided herein are floating hydrogel droplet culture methods that enable scaling of stem cell derived alveolar epithelial cell (AEC) expansion to numbers compatible with large animal or human whole lung engineering, as well as molds for generating the droplets and methods of use thereof.

### CLAIM OF PRIORITY

This application claims the benefit of U.S. Provisional Application Ser. No. 62/927,797, filed on Oct. 30, 2019, and 62/945,834, filed on Dec. 9, 2019. The entire contents of the foregoing are incorporated herein by reference.

### TECHNICAL FIELD

Provided herein are floating hydrogel droplet culture methods that enable scaling of stem cell derived alveolar epithelial cell (AEC) expansion to numbers compatible with large animal or human whole lung engineering, as well as molds for generating the droplets and methods of use thereof.

### BACKGROUND

Induced pluripotent stem cell derived alveolar epithelial cells (iPSC-AECs) are a patient-specific cell source for bio-engineering of human pulmonary epithelium. Disease modeling and therapeutic applications require cost effective and technically feasible differentiation and expansion protocols.

### SUMMARY

Provided herein are floating hydrogel droplet culture methods that enable scaling of stem cell derived alveolar epithelial cell (AEC) expansion to numbers compatible with large animal or human whole lung engineering. Stable cellular phenotype was documented through both culture expansion and biomimetic lung culture. These methods can be used for human scale whole organ lung generation.

Thus provided herein are methods for generating an expanded population of alveolar epithelial cells (AECs). The methods include (a) providing a first population of AECs; (b) mixing the first population of AECs into a hydrogel precursor; (c) allowing or promoting gelation of the hydrogel precursor to form a droplet; and (d) culturing the droplets in suspension in moving media sufficient for expansion of the first population, thereby generating an expanded population of AECs. In some embodiments, after step (b), the methods include transferring the mixture to a mold apparatus as described herein, and then after gelation of the hydrogel precursor in step (c), removing the droplet from the mold apparatus.

In some embodiments, the first population of AECs comprises induced pluripotent stem cell (iPSC)-derived AECs.

In some embodiments, the iPSC-derived AECs are obtained by a method comprising: providing an initial population of iPSC; culturing the iPSC under conditions sufficient for definitive endodermal differentiation, then under conditions sufficient for anteriorized endodermal differentiation, and then under conditions sufficient for ventralized endodermal differentiation, thereby obtaining a population of iPSC-derived AECs.

In some embodiments, the droplet has a maximal diameter of 2-10 mm.

In some embodiments, the hydrogel is a natural or synthetic hydrogel scaffold. In some embodiments, the natural hydrogel scaffold comprises extracellular matrix (ECM), collagen, fibrin, bone sialoprotein, vitronectin, alginate, or laminin. In some embodiments, the synthetic hydrogel scaffold comprises a synthetic polymeric scaffold selected from poly(2-(methacryloyloxy) ethyl dimethyl-(3-sulfopropyl)ammonium hydroxide) (PMEDSAH), polyacrylamide (PAM), poly(sodium 4-stryenesulfonate) (PSS), poly(methyl vinylether-alt-maleic anhydride), and poly(ethylene glycol) (PEG) hydrogels.

In some embodiments, allowing or promoting gelation of the hydrogel comprises providing a temperature, chemical, or light sufficient to initiate crosslinking of the hydrogel scaffold.

In some embodiments, the moving media is spinning or flowing culture.

In some embodiments, the expanded population of AECs comprises cells that express Nkx2.1 and aquaporin 5 (AQP5) or Surfactant Protein C (SPC).

Also provided herein are expanded populations of AECs produced by a method described herein.

Additionally, provided herein are methods for providing a bioartificial lung organ. The methods include oroviding an expanded population of AECs produced by a method described herein; providing a (cell-free) lung tissue matrix (e.g., from a human or pig) including an airway and vasculature; seeding the lung tissue matrix with the expanded population of AECs through the airway, with endothelial cells through the vasculature, and with mesenchymal cells through either one or both of the airway and the vasculature; and maintaining the matrix under conditions sufficient for the formation of a functional epithelium in the airways and functional vasculature. Also provided herein are bioartificial lung organs produced by a method described herein.

Further, provided herein is a mold apparatus, comprising: a mold body comprising a polymeric material, the mold body defining a first cavity and a second cavity, the first and second cavities each having a radius of between 0.5 mm and 5 mm and configured to receive a composition, the mold body further defining a first channel that extends along a longitudinal axis that intersects the first and second cavities, wherein the first channel is defined by a depth dimension configured to limit a volume amount of the composition in the first and second cavities. In some embodiments, the polymeric material is flexible. Also provided is a mold apparatus comprising: a flexible body defining a plurality of cavities, the plurality of cavities forming an array pattern comprising at least first and second rows, wherein each row comprises at least two or more cavities aligned along first and second longitudinal axes, respectively, the first and second longitudinal axes being spaced apart from one another by a separation distance, wherein each cavity is configured to form semi-spherical shaped compositions, and the cavities each have a radius of between 0.5 mm and 5 mm and are configured to receive a composition, wherein the cavities are defined by a depth dimension configured to limit a volume amount of the composition in the first and second cavities.

In some embodiments, the flexible body is formed from a polymeric material

In some embodiments, the flexible material is selected from the group consisting of silicones and polyurethanes. In some embodiments, the polymeric material comprises polydimethylsiloxane (PDMS).

In some embodiments, each cavity (e.g., the bottom of each cavity) is defined by a hemispherically shaped surface. In some embodiments, each cavity is configured to form spherically shape compositions or hemi-spherically shaped compositions. In some embodiments, the first channel extends from one side edge of the mold body to a second, opposite side edge.

In some embodiments, the depth dimension is configured to limit the volume of the composition in each cavity to a maximum volume amount of about 50 μL to about 150 μL.

Also provided herein are methods of forming shaped gel compositions, the method comprising adding a composition to cavities of a mold apparatus as described herein, the composition being a liquid comprising a biologic; forming a plurality of semi-solid or solid compositions in the cavities of the mold; and removing the semi-solid or solid compositions from the cavities of the mold.

In some embodiments, the liquid is a hydrogel precursor and the biologic comprises cells.

In some embodiments, the semi-solid or solid composition is a hydrogel.

In some embodiments, the removing step comprises flexing the body of the mold.

In some embodiments, the semi-solid or solid compositions retain a predetermined shape, e.g., in a spin culture, e.g., for at least 1 day, at least 5 days, or at least 10 days.

In some embodiments, the semi-solid or solid compositions are spherical or semi-spherical.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. MATRIGEL adherent droplet culture method showing cell laden MATRIGEL droplets in a 12-well plate.

FIGS. 2A-F. Comparison of culture groups after iPSC-AEC expansion. (A) Comparison of culture cell yield per Matrigel droplet between the floating droplet method (n=4) and the adherent droplet method (n=4) demonstrating significantly greater cell expansion with the floating droplet culture method. (B) Relative Ki67 gene expression in cells from each droplet culture method immediately after the expansion period showing a significant increase in Ki67 gene expression with the floating droplet culture method (p=0.33). (C) Quantitative PCR comparing relative gene expression of Nkx2.1 and SPC between the culture methods after expansion. The adherent droplet culture (plate culture) demonstrated significantly higher Nkx2.1 gene expression (p=0.041), while there was comparable gene expression of SPC between the culture methods. (D-F) H&E stained images of iPSC-derived alveolar pneumocytes from the floating droplet culture demonstrating spontaneous formation of spherical cellular formations termed alveolar spheres at 4×, 20×, and 40× magnification, respectfully. Data are represented as mean±SEM.

FIGS. 3A-D. Phenotypic comparison of iPSC-AECs after expansion. (A) Flow cytometric analysis of iPSC-AECs from the adherent droplet culture method demonstrating preserved Nkx2.1 and SPC expression. (B) Flow cytometric analysis of iPSC-AECs from the floating droplet culture method demonstrating similar phenotype to the adherent droplet culture cells. (C-D) Flow cytometric analysis of iPSC-AECs from the adherent droplet and floating droplet culture methods, respectfully, demonstrating no appreciable AQP5 expression for either culture condition.

FIGS. 4A-J. Tissue protein expression and histologic appearance after biomimetic lung culture. (A) Nkx2.1 protein expression measured by tissue fluorescence per cell after IHC staining demonstrating no significant difference between lungs in either group (n=5 for each). (B) SPC protein expression measured by tissue fluorescence per cell after IHC staining demonstrating significantly decreased SPC expression in lungs cultured with floating droplet cells (p<0.01). (C) AQP5 protein expression measured by tissue fluorescence per cell after IHC staining demonstrating significantly increased AQP5 expression in lungs cultured with floating droplet cells (p<0.001). (D-F) IHC stained images from lungs cultured with floating droplet cells after the biomimetic lung culture for Nkx2.1, SPC, and AQP5, respectfully. Blue nuclear stain used in each image. (G) Relative Ki67 gene expression in cells from each droplet culture method immediately after the expansion period (from FIG. 2, presented here for direct comparison), on day 6 of biomimetic lung culture, and on day 12 of biomimetic lung culture demonstrating a consistent increase in Ki67 expression as the cells progressed through the biomimetic lung culture period. (H-J) H&E stained images from lungs cultured with floating droplet cells after the biomimetic lung culture at 4×, 20×, and 40× magnification, respectfully. Data are represented as mean±SEM.

FIGS. 5A-D. Conditioned media analysis from the biomimetic lung culture. (A) Change in media bicarbonate during the biomimetic lung culture showing comparable bicarbonate consumption at all time points. (B) Lactate generation observed in the media from the biomimetic lung culture showing comparable lactate generation at all time points. (C) Glucose consumption in the biomimetic lung culture showing comparable glucose consumption at all time points. (D) Cellular metabolic activity measured via resazurin assay at both day 6 and 12 of the biomimetic lung culture demonstrating comparable cellular metabolic activity of cells seeded on lung scaffolds from both culture methods. Data are represented as mean±SEM.

FIGS. 6A-D. Rational for described floating droplet culture method. (A) Comparison of culture cell yield per cell laden Matrigel droplet demonstrating significantly greater cell expansion with the floating droplet culture method without mechanical stimulation compared to a culture with high speed mechanical stimulation (35 RPM stirring, Sp35) but still fewer cells than with moderate mechanical stimulation (see FIG. 3). (B) Flow cytometric analysis of iPSC-derived alveolar pneumocytes after expansion with the adherent droplet culture method demonstrating preserved Nkx2.1 and SPC expression after expansion (included from FIG. 3a for direct comparison). (C) Flow cytometric analysis of iPSC-derived alveolar pneumocytes after expansion with the floating droplet culture method without mechanical stimulation or stirring demonstrating Nkx2.1 and SPC expression after expansion. (D) Flow cytometric analysis of iPSC-derived alveolar pneumocytes after expansion with the floating droplet culture method with high speed (35 RPM) mechanical stimulation demonstrating decreased Nkx2.1 and SPC expression.

FIG. 7 is an exemplary illustration of a method for generation and characterization of alveolar spheres from human iPSCs. In this example, BU3-NGST hiPSC (Nkx2.1-GFP, SPC-TdTomato) cells are used.

FIGS. 8A-8D show an exemplary mold apparatus. Specifically, FIG. 8A provides a perspective view of a mold. FIGS. 8B and 8C provide top and side views of the mold, respectively. FIG. 8C provides a cross-sectional view of the mold.

FIGS. 9A-9F shows images of various examples of a mold apparatus described herein. FIG. 9A shows a flexible 12-well polydimethylsiloxane (PDMS) mold with 100 uL wells, designed for cell laden hydrogel droplet formation for floating culture method. FIGS. 9B-9D provide images of a 96-well mold designed for repeat pipetting and rapid filling; this exemplary 96-well mold for droplet formation is in a 96-well configuration, which is amenable for multichannel pipette use. FIG. 9D shows a liquid gel solidified within a sterile mold. The mold demonstrates 96 droplets, each 100 μL in a 6 mm diameter well. The mold was subsequently placed in a 37° C. incubator for 20 minutes to allow for hydrogel solidification. FIG. 9E shows a hydrogel sphere that has been removed from the mold. FIG. 9F shows a flask with a magnetic stir rod containing cell laden hydrogel floating droplets in cell culture media; the spherical hydrogel droplets maintained spherical shape over 7 days in spin culture.

### DETAILED DESCRIPTION

Currently, more than 1300 patients are awaiting a life-saving lung transplant in the United States (1). Of these 1300 patients, approximately 300 patients will die awaiting a pulmonary transplant (2). The fortunate patients who receive a lung transplant still require intensive immunosuppression, which is associated with significant morbidity (3). Lung bioengineering for augmentation or replacement utilizing patient-specific cell populations have the potential to provide an alternative to donor lungs and solve both donor organ shortage and the need for immunosuppression.

Any therapy that aims to replace gas exchange tissue, be it organ engineering or delivery of a cell therapy, depend on the availability of sufficient numbers of human pulmonary epithelial cells. Billions of distal lung epithelial cells from induced pluripotent stem cells (iPSC) are needed to adequately recellularize whole organ lung constructs. This need could be met utilizing directed differentiation of iPSCs toward lung-lineage committed cells. A recently published protocol has been adapted by our laboratory to reproducibly generate type II alveolar epithelial cells (AECs) via directed differentiation and fluorescent sorting of an iPSC line which has been genetically modified to carry fluorescent lung lineage markers (4-6). The resulting cells form alveolar spheres when encapsulated in adherent 3D Matrigel droplets to yield type II AECs from human iPSCs (iPSC-AECs). Type II pneumocytes secrete surfactant which supports alveolar maintenance via reduction of aqueous surface tension and also serve as a reservoir progenitor cell population for type I pneumocytes which facilitate gas exchange (7).

Perfusion-decellularization of rat or human lungs to generate extracellular matrix (ECM) scaffolds suitable for recellularization with iPSC-AECs was previously reported (8, 9). Upscale of this concept to human lungs requires approximately 10.5 billion epithelial cells (10). The established adherent hydrogel droplet culture (4) limits current capacity for large-scale organ recellularization with respect to culture time and resources.

The methods described herein are straightforward cell culture methods for iPSC-AEC expansion that can be scaled for large animal or human lung bioengineering. Matrigel is known to support differentiation and proliferation of iPSC-AECs, but there are challenges associated with its use (8, 9, 14). Matrigel is a liquid only at cold temperatures and rapidly undergoes a gel transition at 37° C., making it difficult to handle (14). The present method speeds droplet formation while maintaining the three-dimensional droplet structure, in contrast to the previously described method in which each drop takes 90 seconds to form (4). Additionally, the floating droplet method allows for greater cell expansion. This is an improvement on previously described methods for iPSC-AEC culture with reduction in labor and physical material expenditure while increasing cell yield.

The present methods are scalable to a variety of culture sizes. The floating droplet culture vessel or volume of culture medium is easily increased or decreased for differing applications. The culture methods can be automated for large iPSC-derived cell farms for commercial applications. The phenotypic stability of the cells in this floating droplet culture system is important. An obvious concern when expanding iPSC-derived cells is transdifferentiation. Comparable metabolic activity and expression of SPC was demonstrated between cells from the two culture methods, while a significant increase in both the cell culture yield and markers of proliferation (Ki67 expression) was seen in the floating droplet culture. The predilection to spontaneously form alveolar spheroids was also preserved on histological review of the cells from the floating droplet culture. When these cells were seeded on a native ECM biomimetic lung culture, they appropriately populated the distal airways with a columnar type epithelium with comparable metabolic activity as evidenced by the resazurin assay and biochemical markers bicarbonate, lactate, and glucose.

Priming iPSC-AECs for Differentiation

There is no established protocol for reliable differentiation of iPSC-derived type I AECs. Yamamoto et al described a subpopulation of iPSC-derived type I AECs identified incidentally when describing a type II AEC differentiation protocol, but this pertained to a very small portion of the differentiated cells (15). An intriguing finding in the present study is the difference in SPC and AQP5 expression in the biomimetic lung culture tissues.

Methods for Generating Stem Cell-Derived Alveolar Cells

Provided herein are scalable methods for generation of stem cell-derived alveolar cells. The methods include culturing the cells in matigel droplets formed using a method described herein.

The present methods can be performed using a starting population of stem cells, e.g., cells from a human embryonic stem cell line (e.g., H9, H1) or embryonic stem cell-like (ESC-like) induced pluripotent stem cells (iPSCs), e.g., generated from primary cells autologous to a subject to be treated using a method described herein. Primary cells such as airway basal cells, lineage negative lung progenitor cells, club cells or type II pneumocytes can also be used.

Methods for generating iPSC are known in the art. In some embodiments, the methods for generating hiPSC can include obtaining a population of primary somatic cells from a subject, e.g., a subject who is afflicted with PD and in need of treatment for PD. Preferably the subject is a mammal, e.g., a human. In some embodiments, the somatic cells are fibroblasts. Fibroblasts can be obtained from connective tissue in the mammalian body, e.g., from the skin, e.g., skin from the eyelid, back of the ear, a scar (e.g., an abdominal cesarean scar), or the groin (see, e.g., Fernandes et al., Cytotechnology. 2016 March; 68(2): 223-228), e.g., using known biopsy methods. Other sources of somatic cells for hiPSC include hair keratinocytes (Raab et al., Stem Cells Int. 2014; 2014:768391), blood cells, or bone marrow mesenchymal stem cells (MSCs) (Streckfuss-Bömeke et al., Eur Heart J. 2013 September; 34(33):2618-29). In some embodiments, the primary cells (e.g., fibroblasts) are exposed to (cultured in the presence of) factors sufficient to induce reprogramming to iPSC. Peripheral blood-derived mononuclear cells can be isolated from patient blood samples and used to generate induced pluripotent stem cells. In other examples, induced pluripotent stem cells can be obtained by reprograming with constructs optimized for high co-expression of Oct4, Sox2, Klf4, c-MYC in conjunction with small molecule such as transforming growth factor β (SB431542), MEK/ERK (PD0325901) and Rho-kinase signaling (Thiazovivin). See Groß et al., Curr Mol Med. 13:765-76 (2013) and Hou et al., Science 341:651:654 (2013). Methods for generating endothelial cells from stem cells are reviewed in Reed et al., Br J Clin Pharmacol. 2013 April; 75(4):897-906. Cord blood stem cells can be isolated from fresh or frozen umbilical cord blood. Mesenchymal stem cells can be isolated from, for example, raw unpurified bone marrow or ficoll-purified bone marrow. Epithelial and endothelial cells can be isolated and collected from living or cadaveric donors, e.g., from the subject who will be receiving the bioartificial lung, according to methods known in the art. For example, epithelial cells can be obtained from a skin tissue sample (e.g., a punch biopsy), and endothelial cells can be obtained from a vascular tissue sample.

Although other protocols for programming can be used (e.g., as known in the art or described herein), in preferred embodiments the present methods can include introducing (contacting or expressing in the cell) four transcription factors, i.e., Oct4, Sox2, Klf4, and L-Myc, known colloquially as the as Yamanaka 4 factors (Y4F). See, e.g., Takahashi and Yamanaka, Cell. 2006; 126(4):663-676; Takahashi et al., Cell. 2007; 131(5):861-872; Yu et al. Science. 2007; 318(5858):1917-1920; Park et al., Nature. 2008; 451(7175):141-146. In some embodiments, the methods also include contacting or expressing in the cell one or more miRNAs, e.g., (i) at least one miR-302 cluster member and (ii) at least one miR-200 cluster member; see US 20160298089 and Song et al., J Clin Invest. 2020; 130(2):904-920.

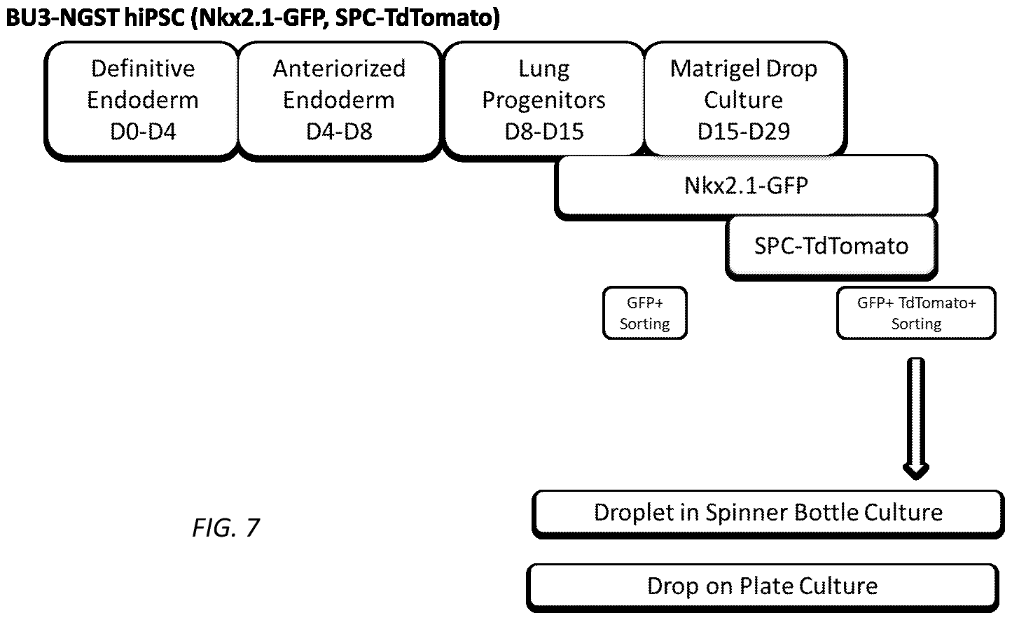

The starting population of stem cells is differentiated to alveolar epithelial cells (AECs) via directed differentiation, e.g., as shown in FIG. 7. First, the cells undergo definitive endodermal differentiation for about 4 days, followed by about 4 days of anteriorized endodermal differentiation in the presence of a TGFb antagonist (e.g., A8301) and BMP antagonist (e.g., IWR-1). The cells then undergo ventralized endodermal differentiation for about 7 days in the presence of growth factors, e.g., fibroblast growth factors, e.g., FGF-7 and FGF-10, and a GSK3 inhibitor/WNT pathway activator (e.g., CHIR99021). Tables A and B below provide a number of alternative protocols for differentiation; the exemplary protocol used in the examples below is indicated as the preferred protocol.

TABLE A
Basal Medium

| | | | | Preferred |
|---|---|---|---|---|
| 1 | 2 | 3 | 4 | Protocol |
| RPMI, B27(-Insulin) | DMEM/F12, N2, | StemDiff for DE | RPMI + 0%, | StemDiff for DE |
| for DE | B27(Complete), | (Stem Cell | 0.2%, 2%, 2% | (Stem Cell |
| RPMI, B27 | Ascorbic Acid | Technology) | dFBS for DE | Technology) |
| (complete) | (50 ug/ml), | Ham's | DMEM/F12, | DMEM/F12 + |
| | Glutamax | F12/IMDM 1:3 | N2, | B27(Complete) |
| | (2 mM), | B27 + RA 0.5x | B27(Complete) | for AFE and VE |
| | Monothioglycero | N2 BSA (0.5 g/ml) | | DMEM/F12:M199 = |
| | (0.4 uM), | Monothioglycero | | 1:1 + B27(Complete) |
| | 0.05% BSA | (3 um/ml) | | FBS(2%) |
| | | Glutamax | | Ascorbic Acid |
| | | Ascorbic Acid | | (50 ug/ml) |
| | | (50 ug/ml) | | for AEC |
| | | | | expansion |

TABLE B
Inductive factors

| | | | | | Preferred |
|---|---|---|---|---|---|
| | 1 | 2 | 3 | 4 | protocol |
| Definitive | 50 ng/ml | Embryoid | StemDiff for DE | 100 ng/ml | StemDiff for DE |
| Endoderm | Activin A | Bodies | (Stem Cell | Activin A | (Stem Cell |
| | 10 uM | 10 μM Rock | Technology) | 0%, 0.2%, | Technology) |
| | ROCK | inhibitor | 4 days | 2%, 2% | 4 days |
| | inhibitor | 3 ng/ml | | dFBS | |
| | 4 days | BMP4 | | 4 days | |
| | | 1 day | | | |
| | | 10 μM Rock | | | |
| | | inhibitor 0.5 | | | |
| | | ng/ml BMP4 | | | |
| | | 1.5 ng/ml | | | |
| | | bFGF | | | |
| | | 100 ng/ml | | | |
| | | Activin A | | | |
| | | 3 days | | | |
| Anteriorize | 1 uM A8301 | 10 μM | 10 μM | 10 μM | 1 um A8301 |
| Endoderm | (TGFb | SB431542 | SB431542 | SB431542 | (TGFb |
| | antagonist) | (TGFb | (TGFb | (TGFb | antagonist) |
| | 0.5 um | antagonist) | antagonist) | antagonist) | 1 um IWR-1 |
| | DMH1 | 1.5 μM | 2 μM | 200 ng/ml | (BMP antagonist) |
| | (BMP antagonist) | Dorsomorphin | Dorsomorphin | Noggin | |
| | 2-4 days | (BMP antagonist) | (BMP antagonist) | (BMP antagonist) | |
| | | 1 day | 3 days | 500 ng/ml | |
| | | 10 μM | | FGF4 | |
| | | SB431542 | | 2 uM | |
| | | (TGFb | | CHIR99021 | |
| | | antagonist) | | | |
| | | 1 μM IWP2 | | | |
| | | (Wnt inhibitor) | | | |
| | | 1 day | | | |
| Ventralization | 100 ng/ml | 10 ng/ml | 10 ng/ml BMP4 | | 10 ng/ml FGF10 |
| | FGF2 | FGF10 | 100 nm ATRA | | 10 ng/ml FGF7 |
| | 10 ng/ml | 10 ng/ml FGF7 | 3 uM | | 10 ng/ml BMP4 |
| | BMP4 | 10 ng/ml | CHIR99021 | | 100 nm ATRA |
| | 100 nM | BMP4 | (GSK3 inhibitor/ | | 3 uM |
| | CHIR99021 | 1 um ATRA | WNT pathway | | CHIR99021 |
| | (GSK3 | 3 uM | activator) | | (GSK3 inhibitor/ |
| | inhibitor/WNT | CHIR99021 | 9 days | | WNT pathway |
| | pathway | (GSK3 inhibitor/ | | | activator) |
| | activator) | WNT pathway | | | 7 days |
| | 4 days | activator) | | | |
| | | 8-10 days | | | |
| Alveolar | | | 10 ng/ml FGF7 | | 10 ng/ml FGF10 |
| Epithelial | | | 3 uM | | 10 ng/ml FGF7 |
| Cell | | | CHIR99021 | | 3 uM |
| Generation | | | 0.1 mM 8camp | | CHIR99021 |
| | | | 0.1 mM IBMX | | 0.1 mM 8camp |
| | | | 50 nM | | 0.1 mM IBMX |
| | | | Dexamethasone | | 50 nM |
| | | | 7-14 days | | Dexamethasone |
| | | | | | 7-14 days |

1: Mou, H., Zhao, R., Sherwood, R., Ahfeldt, T., Lapey, A., Wain, J., Sicilian, L., Izvolsky, K., Lau, F. H., Musunuru, K. and Cowan, C., 2012. Generation of multipotent lung and airway progenitors from mouse ESCs and patient-specific cystic fibrosis iPSCs. Cell stem cell, 10(4), pp. 385-397.
2: Huang, S. X., Islam, M. N., O'neill, J., Hu, Z., Yang, Y. G., Chen, Y. W., Mumau, M., Green, M. D., Vunjak-Novakovic, G., Bhattacharya, J. and Snoeck, H. W., 2014. Efficient generation of lung and airway epithelial cells from human pluripotent stem cells. Nature biotechnology, 32(1), pp. 84-91.
3: Jacob, A., Morley, M., Hawkins, F., McCauley, K. B., Jean, J. C., Heins, H., Na, C. L., Weaver, T. E., Vedaie, M., Hurley, K. and Hinds, A., 2017. Differentiation of human pluripotent stem cells into functional lung alveolar epithelial cells. Cell stem cell, 21(4), pp. 472-488.
4: Dye, B. R., Hill, D. R., Ferguson, M. A., Tsai, Y. H., Nagy, M. S., Dyal, R., Wells, J. M., Mayhew, C. N., Nattiv, R., Klein, O. D. and White, E. S., 2015. In vitro generation of human pluripotent stem cell derived lung organoids. elife, 4, p.e05098.

These steps are carried out using standard culture methods, e.g., in culture dishes. After ventralization, the cells can be fluorescence-activated cell sorted (FACS) for purification; for example, in cells that express a reporter protein, that reporter protein can be used (exemplified herein is the sorting of Nkx2.1-GFP positive cells).

The cells are then mixed in a natural or synthetic hydrogel scaffold, e.g., comprising natural extracellular matrix (ECM), e.g., MATRIGEL (Corning, Corning, N.Y.), GELTREX LDEV-Free Reduced Growth Factor Basement Membrane Matrix (GIBCO/ThermoFisher), or CULTREX Basement Membrane Extract (BME) (Trevigen); natural scaffolds comprising collagen (e.g., and collagen type IV), fibrin, bone sialoprotein, vitronectin (e.g., VITRONECTIN XF™ (STEMCELL Technologies), alginate, or laminin; synthetic polymeric scaffolds, e.g., comprising poly(2-(methacryloyloxy) ethyl dimethyl-(3-sulfopropyl)ammonium hydroxide) (PMEDSAH), polyacrylamide (PAM), poly(sodium 4-stryenesulfonate) (PSS), poly(methyl vinylether-alt-maleic anhydride), or poly(ethylene glycol) (PEG) hydrogels (e.g., photo-crosslinked or enzymatically crosslinked PEG-vinyl sulfone (PEG-VS), photopolymerizable PEG thiol-ene hydrogel scaffolds with cysteine-flanked MMP-sensitive crosslinks, or MMP-degradable, RGD-functionalized PEG hydrogel scaffolds factor-Mk-mediated crosslinked peptide-functionalized PEG monomers), or combinations thereof. In some embodiments, additional factors are included such as heparin sulfate proteoglycans such as perlecan, or peptides are used to promote cell growth or adhesion to synthetic or natural scaffolds, e.g., laminin-derived peptide (YIGSR) or fibronectin-derived Arg-Gly-Asp (RGD) peptides, linear or circularized (cyclo(Arg-Gly-Asp-d-Phe-Lys) (cRGDfK)), e.g., SYNTHEMAX, a synthetic vitronectin scaffold functionalized with RGD (Corning). A number of suitable scaffolds are known in the art. See, e.g., Cruz-Aculia and Garcia, Matrix Biol. 2017 January; 57-580:324-333; Murrow et al., Development. 2017; 144:998-1007; Murphy et al., Nat Mater. 2014; 13:547-557; Nguyen et al., Nat Biomed Eng. 2017; 1: 0096; and Aisenbrey and Murphy, Nature Reviews Materials 5:539-551 (2020), and references cited therein. In some embodiments, the hydrogel scaffold composition comprises one or more growth factors, e.g., VEGF, FGF (e.g., bFGF), TGFbeta inhibitors, kir, Wnt inhibitors,

The cells are mixed into a hydrogel scaffold precursor (e.g., in liquid or semi-liquid form, i.e., sufficiently flowable to be easily transferred), and then the mixture is transferred to a droplet mold as described herein, and gelling is allowed or promoted, e.g., by initiation of crosslinking as appropriate to the selected hydrogel scaffold. The hydrogel has an elasticity and shear modulus (stiffness) sufficient to retain the shape of a formed droplet.

The droplets are three-dimensional. In some embodiments, the droplets are substantially spherical, ovoid, cylinder, cube, or cuboid. In some embodiments, the volume of the droplet is about 50-150 μL. In some embodiments, the droplets are 1-10 mm in diameter or width, e.g., 3-9 mm, 5-7 mm, or about 6 mm. In some embodiments, the droplets each comprise about 1,000-50,000 cells, e.g., about 10,000-30,000, e.g., about 20,000 cells.

After gelation the droplet is removed from the mold and placed into a suspension culture, e.g., a spinning or flowing culture, in media sufficient to support expansion of the cells. The droplets can be maintained in culture long enough to allow for proliferation (expanstion) of the cell population to desired levels.

### Methods of Use

The expanded populations of cells can be used, e.g., in transplantation protocols. The cells can be transplanted directly, or can be used to recellularize whole or partial organ lung constructs. Methods for making lung constructs are known in the art; see, e.g., US 20170326273; US 20170073645; U.S. Pat. No. 10,624,992.

For example, the cells can be used to seed a lung tissue matrix, e.g., introduced into the matrix through the airway (tracheal) line (epithelial cells). For example, a tissue matrix can be seeded with the expanded AECs in vitro at any appropriate cell density. In addition, a matrix comprising an airway and vasculature can be seeded with the AECs through the airway, with endothelial cells through the vasculature, and with mesenchymal cells through either one or both of the airway and the vasculature. For example, cell densities for seeding a matrix can be at least 1×10³ cells/gram matrix. Cell densities can range between about 1×10⁵ to about 1×10¹⁰ cells/gram matrix (e.g., at least 100,000, 1,000,000, 10,000,000, 100,000,000, 1,000,000,000, or 10,000,000,000 cells/gram matrix) can be used.

In some cases, a decellularized or artificial lung tissue matrix, as provided herein, can be seeded with the cell types and cell densities described above, e.g., by gravity flow or perfusion seeding. For example, a flow perfusion system can be used to seed the decellularized lung tissue matrix via the vascular system preserved in the tissue matrix (e.g., through the arterial line). In some cases, automated flow perfusion systems can be used under the appropriate conditions. Such perfusion seeding methods can improve seeding efficiencies and provide more uniform distribution of cells throughout the composition. Quantitative biochemical and image analysis techniques can be used to assess the distribution of seeded cells following either static or perfusion seeding methods.

In some cases, a tissue matrix can be impregnated or perfused with one or more growth factors to stimulate expansion of the seeded cells. For example, a tissue matrix can be impregnated or perfused with growth factors appropriate for the methods and materials provided herein, for example, vascular endothelial growth factor (VEGF), TGF-growth factors, bone morphogenetic proteins (e.g., BMP-1, BMP-4), platelet-derived growth factor (PDGF), basic fibroblast growth factor (b-FGF), e.g., FGF-10, insulin-like growth factor (IGF), epidermal growth factor (EGF), or growth differentiation factor-5 (GDF-5). See, e.g., Desai and Cardoso, Respire. Res. 3:2 (2002). These growth factors can be encapsulated to control temporal release. Different parts of the scaffold can be enhanced with different growth factors to add spatial control of growth factor stimulation. In the present methods, the cells seeding the airway can be perfused with a notch inhibitor, e.g., a gamma secretase inhibitor.

Seeded tissue matrices can be incubated for a period of time (e.g., from several hours to about 14 days or more) post-seeding to improve adhesion and penetration of the cells in the tissue matrix. The seeded tissue matrix can be maintained under conditions in which at least some of the regenerative cells can multiply and/or differentiate within and on the acellular tissue matrix. Such conditions can include, without limitation, the appropriate temperature (35-38 degree centigrade) and/or pressure (e.g., atmospheric), electrical and/or mechanical activity (e.g., ventilation via positive or negative pressure with positive end expiratory pressure from 1-20 cmH2O, mean airway pressure from 5-50 cmH2O, and peak inspiratory pressure from 5-65 cmH2O), the appropriate gases, e.g., O₂ (1-100% FiO₂) and/or CO₂ (0-10% FiCO2), an appropriate amount of humidity (10-100%), and sterile or near-sterile conditions. Such conditions can also include wet ventilation, wet to dry ventilation and dry ventilation. In some cases, nutritional supplements (e.g., nutrients and/or a carbon source such as glucose), exogenous hormones, or growth factors can be added to the seeded tissue matrix. In preferred embodiments, a notch inhibitor, e.g., a gamma secretase inhibitor, is added to the cells seeded through the airway (see, e.g., U.S. Pat. No. 10,624,992). Histology and cell staining can be performed to assay for seeded cell retention and propagation. Any appropriate method can be performed to assay for seeded cell differentiation. In general, the methods described herein will be performed in an airway organ bioreactor apparatus, e.g., as described herein.

Thus, the methods described herein can be used to generate a transplantable bioartificial lung tissue, e.g., for transplanting into a human subject. As described herein, a transplantable tissue will preferably retain a sufficiently intact vasculature that can be connected to the patient's vascular system.

The bioartificial lung tissues described herein can be combined with packaging material to generate articles of manufacture or kits. Components and methods for producing articles of manufacture are well known. In addition to the bioartificial tissues, an article of manufacture or kit can further can include, for example, one or more anti-adhesives, sterile water, pharmaceutical carriers, buffers, and/or other reagents for promoting the development of functional lung tissue in vitro and/or following transplantation. In addition, printed instructions describing how the composition contained therein can be used can be included in such articles of manufacture. The components in an article of manufacture or kit can be packaged in a variety of suitable containers.

The entire disclosures of all of the foregoing are hereby incorporated by reference herein.

Methods for Using Bioartificial Lungs

This document also provides methods and materials for using bioartificial lung tissues and, in some cases, promoting lung function. In some embodiments, the methods provided herein can be used to restore some lung function in patients having diseases that impair or reduce lung capacity (e.g., cystic fibrosis, COPD, emphysema, lung cancer, asthma, pulmonary hypertension, lung trauma, or other genetic or congenital lung abnormalities, e.g., bronchogenic cyst, pulmonary agenesis and hypoplasia, polyalveolar lobe, alveolocapillary dysplasia, sequestration including arteriovenous malformation (AVM) and scimitar syndrome, pulmonary lymphangiectasis, congenital lobar emphysema (CLE), and cystic adenomatoid malformation (CAM) and other lung cysts). The methods provided herein also include those wherein the subject is identified as in need of a particular stated treatment, e.g., increased lung function, or increased or improved lung capacity.

Bioartificial lung tissues (e.g., whole organs or portions thereof) can be generated according to the methods provided herein. In some embodiments, the methods comprise transplanting a bioartificial lung tissue as provided herein to a subject (e.g., a human patient) in need thereof. In some embodiments, a bioartificial lung tissue is transplanted to the site of diseased or damaged tissue. For example, bioartificial lung tissues can be transplanted into the chest cavity of a subject in place of (or in conjunction with) a non-functioning or poorly-functioning lung; methods for performing lung transplantation are known in the art, see, e.g., Boasquevisque et al., Surgical Techniques: Lung Transplant and Lung Volume Reduction, Proceedings of the American Thoracic Society 6:66-78 (2009); Camargo et al., Surgical maneuvers for the management of bronchial complications in lung transplantation, Eur J Cardiothorac Surg 2008; 34:1206-1209 (2008); Yoshida et al., “Surgical Technique of Experimental Lung Transplantation in Rabbits,” Ann Thorac Cardiovasc Surg. 11(1):7-11 (2005); Venuta et al., Evolving Techniques and Perspectives in Lung Transplantation, Transplantation Proceedings 37(6):2682-2683 (2005); Yang and Conte, Transplantation Proceedings 32(7):1521-1522 (2000); Gaissert and Patterson, Surgical Techniques of Single and Bilateral Lung Transplantation in The Transplantation and Replacement of Thoracic Organs, 2d ed. Springer Netherlands (1996).

The methods can include transplanting a bioartificial lung or portion thereof as provided herein during a surgical procedure to partially or completely remove a subject's lung and/or during a lung resection. The methods can also include harvesting a lung or a portion thereof from a live donor or cadaver and preserving or regenerating the lung in a bioreactor described herein. In some cases, the methods provided herein can be used to replace or supplement lung tissue and function in a subject, e.g., a human or animal subject.

Any appropriate method(s) can be performed to assay for lung function before or after transplantation. For example, methods can be performed to assess tissue healing, to assess functionality, and to assess cellular in-growth. In some cases, tissue portions can be collected and treated with a fixative such as, for example, neutral buffered formalin. Such tissue portions can be dehydrated, embedded in paraffin, and sectioned with a microtome for histological analysis. Sections can be stained with hematoxylin and eosin (H&E) and then mounted on glass slides for microscopic evaluation of morphology and cellularity. For example, histology and cell staining can be performed to detect seeded cell propagation. Assays can include functional evaluation of the transplanted tissue matrix or imaging techniques (e.g., computed tomography (CT), ultrasound, or magnetic resonance imaging (e.g., contrast-enhanced MRI)). Assays can further include functional tests under rest and physiologic stress (e.g., body plethysmography, lung function testing). Functionality of the matrix seeded with cells can be assayed using methods known in the art, e.g., histology, electron microscopy, and mechanical testing (e.g., of volume and compliance). Gas exchange can be measured as another functionality assay. To assay for cell proliferation, thymidine kinase activity can be measured by, for example, detecting thymidine incorporation. In some cases, blood tests can be performed to evaluate the function of the lungs based on levels of oxygen in the blood.

To facilitate functionality assays during culture, any line of the bioreactor apparatus' described herein may include sampling ports to allow for single or real-time measurements of functionality parameters (e.g., pH, glucose, lactate, Na, K, Ca, Cl, bicarb, O₂, CO₂, sat). Metabolites may also be used to monitor cell number and viability using colorimetric assays, and biochemical assays may be used to monitor cell maturation (e.g., measuring surfactant protein, etc.) For example, an increased concentration of surfactant can indicate that the culture lung possesses sufficient epithelial cells to withstand dry ventilation. In some cases, endothelial barrier function may be used as a marker of vascular maturity. Lungs can be perfused with different sizes of molecules (such as dextrans of defined sizes and albumin), and microbeads (increasing sizes from 0.2 to 5 um), as well as isolated red blood cells. Bronchoalveolar lavage fluid can then be sampled to assess leakage of these markers into the alveolar space. For example, 500-kDa dextran can be used in combination with a Bronchoalvelar lavage assay to determine the percentage of dextran retained within the vascular compartment. An increase in the percentage of dextran retained indicates an improvement in the barrier function because barrier function to dextran is dependent on viable and functional endothelium, while dextran will diffuse across a denuded vascular basement membrane (e.g., in an acellular lung) over time during constant perfusion. For example, a cadaveric lung may retain substantially all of the dextran within the vascular compartment while acellular lungs may retain a small percentage of the dextran (e.g., 10.0%±8.0%). Leakage of these markers into the alveolar space greater than a tolerated minimum (for example >10% of 4 um microbeads, or greater than 20% of 0.2 um microbeads) can be used to indicate that the lung is not sufficiently mature to withstand dry ventilation.

In some cases, molecular biology techniques such as RT-PCR can be used to quantify the expression of metabolic (e.g. surfactant protein, mucin-1) and differentiation markers (e.g. TTF-1, p63, surfactant protein C). Any appropriate RT-PCR protocol can be used. Briefly, total RNA can be collected by homogenizing a biological sample (e.g., tendon sample), performing a chloroform extraction, and extracting total RNA using a spin column (e.g., RNeasy® Mini spin column (QIAGEN, Valencia, Calif.)) or other nucleic acid-binding substrate. In other cases, markers associated with lung cells types and different stages of differentiation for such cell types can be detected using antibodies and standard immunoassays.

### Airway Organ Bioreactor Apparatus

An exemplary airway organ bioreactor and methods of use thereof are described in WO 2015/138999, which is incorporated herein by reference in its entirety. Other exemplary bioreactors are described in Charest et al., Biomaterials. 2015 June; 52:79-87. doi: 10.1016/j.biomaterials.2015.02.016; Gilpin et al., Ann Thorac Surg. 2014 November; 98(5):1721-9; discussion 1729. doi: 10.1016/j.athoracsur.2014.05.080; Price et al., Tissue Eng Part A 2010; 16(8):2581-91; Petersen et al., Cell Transplant 2011; 20(7):1117-26; Bonvillain et al., J Vis Exp 2013; (82):e50825; Nichols et al., J Tissue Eng Regen Med. 2016 Jan. 12. doi: 10.1002/term.2113.

### Mold Apparatus

Provided herein is a mold apparatus configured for forming a plurality of shaped solid or semi-solid compositions containing a biologic (e.g., cells). For example, a mold body having multiple cavities (e.g., wells) described herein can be configured for high-throughput formation of semi-solid or solid biological compositions (e.g., gels), such as cell-laden hydrogel droplets as described herein. The body of the mold apparatus described herein can be made of a flexible material that advantageously allows the mold to be flexed, which in turn, facilitates the release of molded compositions within the cavities. In some cases, the mold apparatus described herein can be made of materials that are biocompatible to advantageously produce shaped materials without introducing components that can cause an adverse reaction in a subject. In some cases, the mold apparatus is made of materials having chemical, thermal and/or mechanical properties capable of withstanding stress or thermal-incurring processes, such as sterilization (e.g., using an autoclave process).

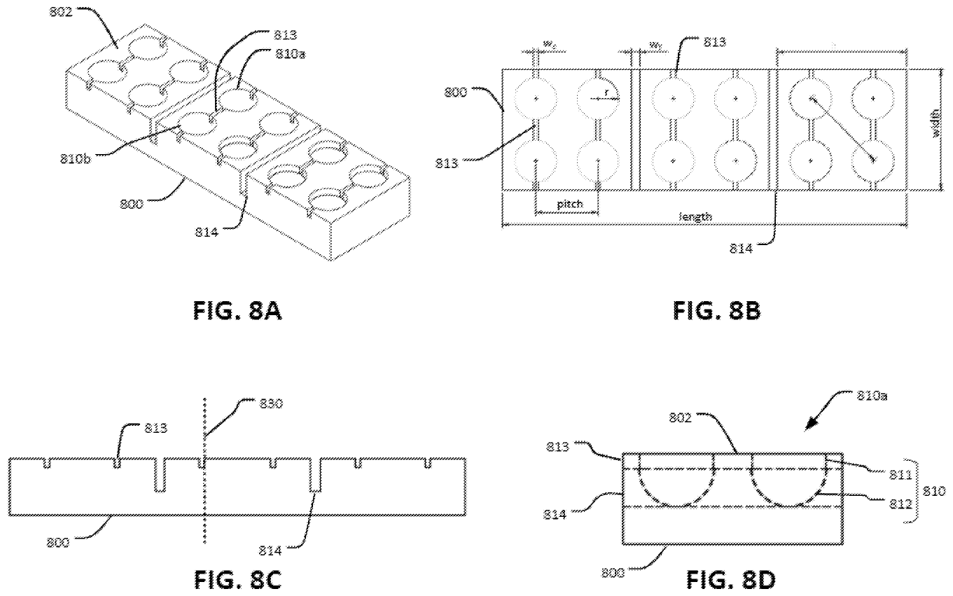

FIGS. 8A-8D show an example of a mold apparatus 800 provided herein. FIG. 8A provides a perspective view of mold apparatus 800. The overall shape of mold apparatus 800 of FIG. 8A is a rectangular-shaped block that includes multiple cavity units or cavities (also referred to herein as “wells”). The block has length, width, and height dimensions that can be adjusted as needed. In some cases, the overall shape of the mold apparatus can be formed into a variety of shapes, such as a polygonal (e.g., square shape) or a curved (e.g., ovoid or spherical) shape.

In some embodiments, the mold apparatus 800 is composed of one or more materials that are heat-resistant. For example, in some cases, the mold can be made of one or more materials that do not plastically deform at temperatures of about 135° C. or more (e.g., about 121° C. or more, about 127° C. or more). In some embodiments, the mold apparatus is made of one or more materials that is pressure-resistant. For example, in some cases, the mold can be made of materials that do not plastically deform when subjected to pressures of about 15 psi or more (e.g., about 10 psi or more, or about 12 psi or more). The mold can be made of materials that advantageously allow the mold to be compatible with thermal or chemical sterilization processes, such as an autoclave or other sterilization process. In some embodiments, the mold can be made of materials that are biologically and/or chemically inert.

In various embodiments, the mold provided herein can be a flexible mold; alternatively, the mold can be rigid. In some embodiments, the materials of the mold can be include one or more polymers. In some embodiments, the materials can include one or more elastomers. In some embodiments, the material is polyurethane, e.g., thermoplastic polyurethane (TPU). In some embodiments, the material is silicone or silicon-based, e.g., polydimethylsiloxane (PDMS). In some embodiments, the material is Poly(methyl methacrylate) (PMMA), Polycarbonate, Polystyrene, Poly(ethylene glycol) diacrylate (PEGDA), Cyclic Olefin Copolymer (COP), or Cyclic Olefin Polymer (COP). Where the mold is rigid, alternative methods can be used to remove the droplets from the wells, e.g., inclusion of a narrow channel running from the bottom of each well to the back or bottom of the mold, allowing for insertion of a wire, needle or plunger to push the droplets from the wells, or for introduction of air pressure to blow the gelled droplets from the wells.

Still referring to FIGS. 8A-8D, the mold apparatus 800 includes a number of hemispherical wells 810 constructed into a first surface 802 of the mold apparatus 800 (e.g., an upper surface). The wells 810 can be arranged in parallel rows along the length of the upper surface 802. For example, as shown, mold apparatus 800 of FIG. 8A includes two parallel rows of six wells each, totaling twelve wells 810. Any number of wells can be included, e.g., 12, 48, 96, and so on; the number of wells can be selected to match an automated aliquotting device that is used to introduce the hydrogel precursor and cells into the wells. A linear channel 813 can span the upper surface 802 of the mold apparatus 800 connecting the edges of the upper surface 802 and bisecting the wells 810 of each row, connecting each well 810 to adjacent wells 810 in adjacent rows. In some embodiments, well 810a is connected to well 810b and the upper right edge of mold 800 is connected to the left edge via channel 813. The channel 813 can extend from the upper surface 802 of the mold 810 to a depth that is less than the depth of the well 810 such that when liquid is disposed into a well 810, the liquid fills each well 810 to a maximum height corresponding to the difference between the depth of the well 810 and the depth of channel 813. Liquid in excess of this maximum height can flow along channel 813 either to a connected well 810, or to the edge of the mold 800. In this manner, each well 810 can advantageously be filled with approximately the same amount of liquid, creating a plurality of droplets of approximately the same size. Materials (e.g., a liquid composition comprising cells and hydrogel precursor) can be deposited into the wells 810 and solidify (e.g., by gelling, polymerization) into a semi-solid or solid form that is shaped by interior walls of the well 810. In some cases, materials can be deposited into a hemispherical well and formed into a hemispherical shape following solidification of the material. The wells 810 of mold apparatus 800 can be shaped in a variety of different shapes including geometric or curvilinear shapes. For example, the wells 810 can be shaped in a cube, a cylinder, rectangular prism, a hemi-ovoid, or a hemi-elliptical shape.

The example mold apparatus 800 of FIG. 8A further includes trenches 814 running parallel with the channels 813 and separating the wells 810 into subgroups. For example, mold apparatus 800 includes two trenches 814 separating the twelve wells 810 into four subgroups of four wells 910 each. The trenches 814 have a greater depth and width than channels 813 and extend from one side surface of the mold apparatus 800 to the opposing side surface. The trenches 814 aligned perpendicular to the longitudinal axis of mold 800 provide flexibility along the same axis.

The dimensions of mold apparatus 800 or components thereof can be adjusted as desired. For example, in some cases, the dimensions of mold apparatus 800 can range in a millimeter or centimeter scale. For example, the longitudinal length and width of mold apparatus 800 can range from about 10 mm to about 100 mm, up to about 10-20 cm. In some cases, the length of the mold can range from about 30 mm to about 60 mm and the width can range from about 10 to about 40 mm. In some cases, the length of the mold can range from about 8 cm to about 10 cm and the width can range from about 4 cm to about 8 cm. The width of channel 813, w_{c}, can range from about 0.1 mm to about 3 mm (e.g., from about 0.5 to about 2 mm, or from about 0.1 to about 1 mm) and the width of trench 814, w_{T}, can range from about 0.1 mm to about 3 mm (e.g., from about 0.5 to about 1 mm, from about 1 mm to about 2 mm, or from about 0.75 to about 1.5 mm). Trenches 814 of mold apparatus 800 can spaced apart from a respective end of mold 800 by about 10 mm to about 25 mm. Each well 810 of mold apparatus 800 can have the same transverse dimension (e.g., radius). The radius, r, can be between 0.5 mm and 5 mm, e.g., between 2 mm and 4 mm (e.g., between 2.5 mm and 4 mm, between 3 mm and 4 mm, between 3.5 mm and 4 mm, between 2 mm and 3.5 mm, between 2 mm and 3 mm, or between 2 mm and 2.5 mm). The central axis of a well can be spaced apart from a central axis of an adjacent well (e.g., center to center distance, a separation distance, or a pitch) by about 5 to about 20 mm. In some embodiments, each well 810 of mold apparatus 800 can have a depth that is roughly equivalent to the diameter of the well 810 (e.g., 2×r), e.g., about 0.5-5 mm, optionally plus the depth of the channel 813.

Still referring to FIGS. 8A-8D, mold apparatus 800 can include channels 813 extending through the width of mold 800. In some embodiments, mold apparatus 800 can also optionally include trenches 814 that extend from an upper surface 802 to a desired depth that is less than the overall height of the mold apparatus 800. In some embodiments, trenches 814 can extend from the upper surface 802 into the mold body to a depth that is approximately equal to the depth of the wells 810. Trenches 814 can advantageously provide elongate flex lines extending in a parallel direction along select rows of the wells. Mold 800 can be compressed flexed in a direction transverse to the trenches to assist with releasing compositions contained within the wells (e.g., separating a gel surface from surfaces of the mold) and facilitate the subsequent removal of the compositions from the wells. In some cases, mold 800 can be flexed to disengage the compositions within the walls of the well, and flipped upside down to facilitate removal of the compositions from mold 800.

FIG. 8D provides a cross-sectional profile of mold apparatus 800 along line 130 shown in FIG. 8C. As shown, hemispherical wells 810a of mold apparatus 800 extend into the upper surface 802 of mold 800 and are constructed in two geometric portions—an upper portion 811 and a lower portion 812. The upper portion 811 is cylindrically shaped in the section of the mold where the well 810 intersects with the channel 813. The lower portion 812 is hemispherically shaped below the channel 813 (although not shown, in some embodiments, the lower portion below channel 813 can include a cylindrically shaped portion above the hemispherically shaped portion). Trench 814 can extend into the upper surface 802 of mold apparatus 800 to a depth about equal to the wells 810. The radius of the cylindrical upper portion 811 of the wells 810 can be approximately equal to the radius of the lower, hemispherical portion 812 of the wells 810. The wells, e.g., the portion of the wells 810 below the intersection with the channel 810, comprising hemispherical portion 812 and optionally part of a cylindrical portion 811, can have an interior volume of about 50 μL to about 150 μL (e.g., of about 60 μL to about 150 μL, of about 80 μL to about 150 μL, of about 800 μL to about 150 μL, of about 120 μL to about 150 μL, of about 140 μL to about 150 μL, of about 50 μL to about 140 μL, of about 50 μL to about 120 μL, of about 50 μL to about 800 μL, of about 50 μL to about 80 μL, or of about 50 μL to about 60 μL).

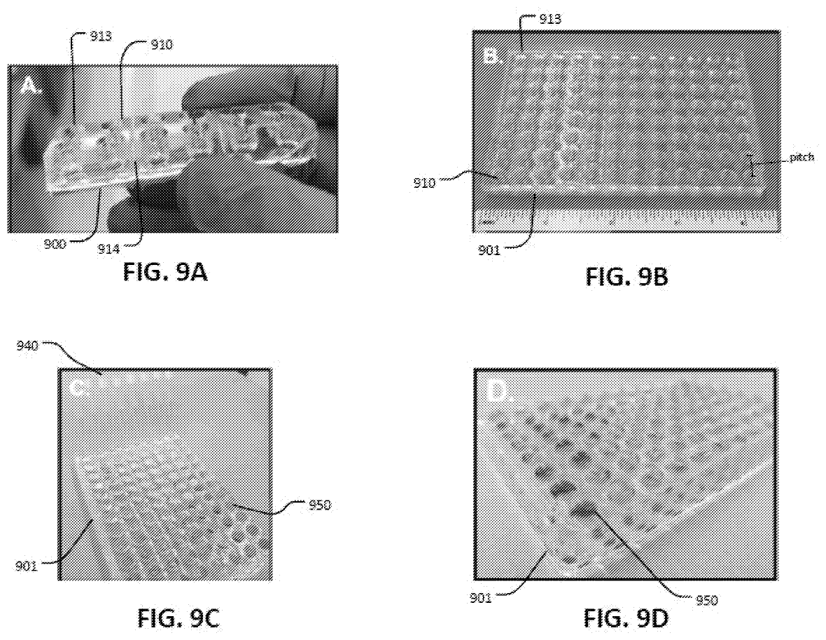

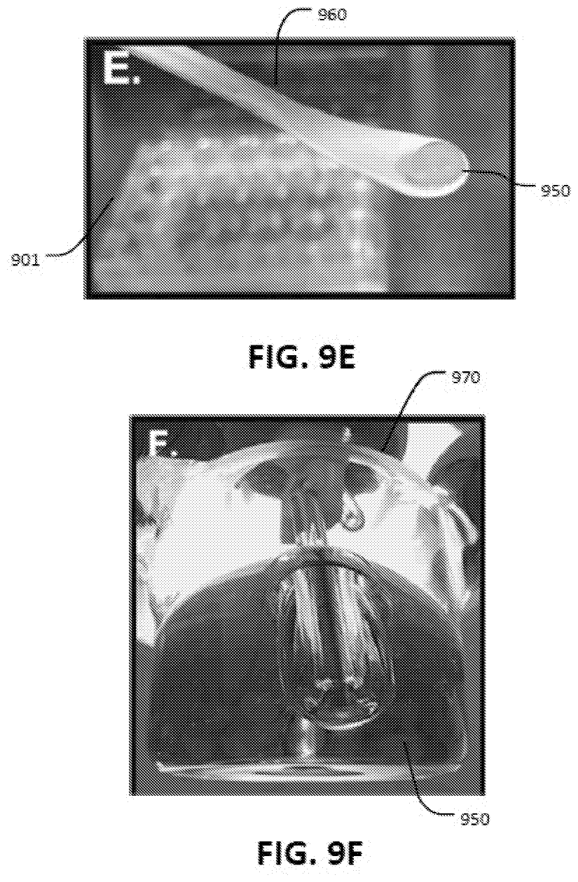

FIGS. 9A-9F shows images of various examples of a mold apparatus described herein. As noted above, a mold can be constructed with different dimensions and a varying number of wells. For example, FIG. 9A shows a mold 900 composed of PDMS that includes 12 wells 910 having an interior volume of about 100 uL. In another example, FIG. 9B shows a mold 901 having a rectangular block that includes 96 wells 910. In some embodiments, the mold can optionally include trenches between select parallel rows of wells (as shown in FIG. 9A as element 914). The wells 910 can be configured with a pitch depth that can accommodate a multi-channel pipettor, e.g., between 9 mm and 14 mm. The pitch of the wells 910 can be configured to allow for repeat pipetting and rapid filling.

The mold apparatus described herein can optionally define additional apertures configured to receive pipettes. For example, as shown in FIG. 9C, the example mold apparatus 901 is designed to receive and hold eight pipette tips 940 in alignment with the rows of wells 910 at one end of the mold 901.

The molds described herein can be used to form semi-solid or solid shaped compositions using the following steps. The wells 910 of the mold apparatus 901 (see FIG. 9B) can be filled with a liquid composition 950, as shown in FIG. 9C. After filing the wells 910, the composition 950 is allowed a predetermined amount of time to solidify and form into a semi-solid or solid composition, such as a gel (e.g., a hydrogel). In some embodiments, the filled mold can be incubated for a predetermined time. Once the composition 950 is solidified, the composition 950 can be formed into a desired shaped form. In some cases, the composition 950 can solidify into a semi-spherical or spherical shape. In some cases, the composition 950 can form a hydrogel sphere. The mold 901 of FIG. 9C is shown after a volume of composition 950 has been disposed into 88 wells. In some embodiments, the gel material is a hydrogel scaffold as described herein (e.g., MATRIGEL), as shown in FIGS. 9C-9F.

In some embodiments, the composition 950 can be dislodged from the wells 910 by flexing the mold apparatus 910, thereby deforming the well 910 shape and dislodging the composition 950.

In some embodiments, as shown in FIG. 9E, an extraction tool 960 can be used to remove a solidified composition 950 from the mold apparatus 901. The solidified composition 950 is shown retaining a spherical shape after removal from a hemispherical well. In some cases, the solidified composition 950 can maintain its shape for a specific time after being removed from the molds. In some cases, the solidified composition 950 can be subjected to further processing, such as being exposed to a spin culture for a given time period, e.g., as described herein. As shown in FIG. 9F, MATRIGEL compositions 950 were able to maintaining a spherical shape after 7 days in a spin culture. In some embodiments, the compositions 950 can retain their pre-determined shape for at least one day or more (e.g., five days or more, or ten days or more).

### EXAMPLES

The invention is further described in the following examples, which do not limit the scope of the invention described in the claims.

### Materials and Methods

The following materials and methods were used in the examples below, unless otherwise noted.

iPSC Differentiation

BU3 iPSC lines carrying the Nkx2.1-GFP and SPC-TdTomato reporters were obtained from Darrell N. Kotton, M.D. (4, 5). These cells were derived from a donor without known genetic abnormalities (11). This cell line had a normal karyotype by G-banding both before and after gene editing (5).

The differentiation of iPSCs was performed following previously published methods with modifications (4, 12). Briefly, BU3-NGST iPSCs that carry two fluorescent reporters for a lung epithelial progenitor marker and an alveolar type 2 cell marker, Nkx2.1-GFP and Surfactant Protein C (SPC)-TdTomato, respectfully, were maintained in mTESR medium (Stemcell Technologies, Vancouver, Canada). A stepwise differentiation procedure that mimics the lung developmental stages was initiated when cells reached 60-70% confluence. The basal medium for all differentiation steps was Dulbeco's Modified Eagle's Medium (DMEM)/F21 (Gibco, Waltham, Mass.) supplemented with B-27 (Gibco, Waltham, Mass.). First, the cells underwent definitive endodermal differentiation using the StemDiff kit (Stemcell Technologies, Vancouver, Canada) for 4 days followed by 4 days of 1 μM A8301 (Sigma, St. Louis, Mo.) and 1 μM IWR-1 (Sigma, St. Louis, Mo.) for anteriorized endodermal differentiation. The cells then underwent ventralized endodermal differentiation by exposing them to 10 ng/mL FGF-7 (Peprotech, Rocky Hill, N.J.), 10 ng/mL FGF-10 (Peprotech, Rocky Hill, N.J.), and 3 μM CHIR99021 (Tocris, Bristol, UK) for 7 days. After ventralization, the cells were stained with DAPI (Sigma, St. Louis, Mo.) and fluorescence-activated cell sorted (FACS) for purification of Nkx2.1-GFP positive cells.

Sorted Nkx2.1+ cells were embedded in 100% Matrigel (Corning, Corning, N.Y.) drops for the formation of alveolar spheres. Homogenous liquid precursor was aliquoted in 100 μL drops onto 12-well plastic culture plates. The culture medium for the formation, maintenance, and expansion (expansion media) of the alveolar spheres had the following composition: 50% Medium 199 (Life Technologies, Carlsbad, Calif.), 49% DMEM/F12 (Life Technologies, Carlsbad, Calif.), 2% fetal bovine serum (FBS) (Hyclone, Logan, Utah), B-27 (Life Technologies, Carlsbad, Calif.), 10 ng/mL FGF-7, 10 ng/mL FGF-10, 3 μM CHIR99021, 0.1 mM IBMX (Sigma, St. Louis, Mo.), 0.1 mM 8-Bromo-cAMP (Sigma, St. Louis, Mo.), 50 nM dexamethasone (Sigma, St. Louis, Mo.), 10 μM Y-27632 (Cayman Chemical, Ann Arbor, Mich.), and 50 μg/mL ascorbic acid (Stemcell Technology, Vancouver, Canada). The droplets were cultured for 7-14 days followed by Matrigel droplet digestion with Dispase (Corning, Corning, N.Y.). The remaining cellular spheres were trypsinized and FACS for GFP+TdTomato+ cells. These iPSC-AECs were used for further expansion.

Cadaveric Rat Lungs

All animal studies were approved by the Massachusetts General Hospital Institutional Animal Care and Use Committee Protocol #2014N000261 and conducted in accordance with The Guide for the Care and Use of Laboratory Animals. Rat lungs were explanted from outbred adult male Sprague-Dawley rats (300-400 g, Charles River Laboratories, Wilmington, Mass.). All rats were pair housed and given unrestricted access to chow and water prior to use. Animals were anesthetized with 5% isofluorane, a laparotomy was performed, heparin was administered intravascularly via the inferior vena cava, and the animal was sacrificed via exsanguination according to approved protocols. A sternotomy was then performed, and the lungs were explanted as previously described (13).

Floating Droplet Cell Culture

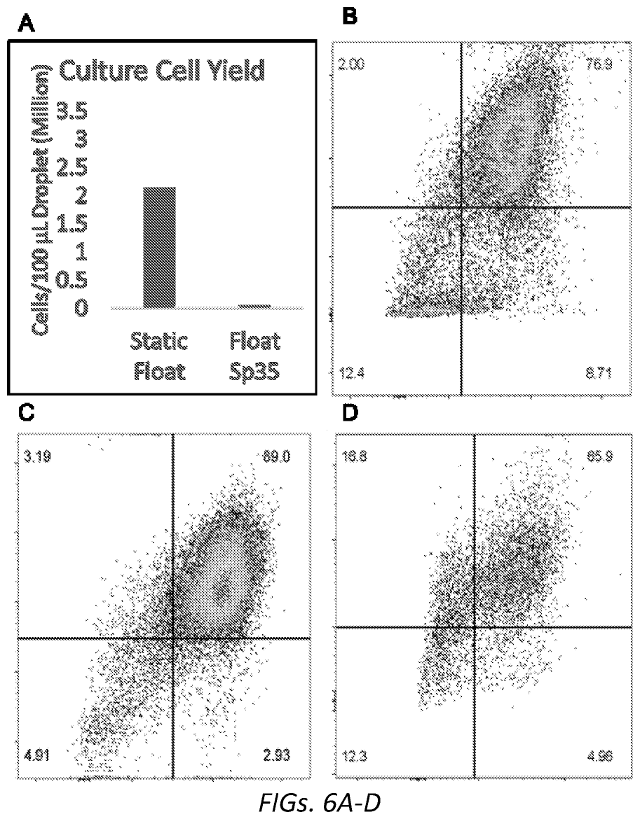

Matrigel with suspended cells was aliquoted into 100 μL drops containing approximately 20,000 cells each. Drops were placed in custom polydimethylsiloxane (PDMS) (Sigma, St. Louis, Mo.) molds (FIG. 9A, C-D) and allowed to gel at 37° C. for 20 minutes. The gelled, cell-laden spheroids were transferred to a magnetic spinner flask which was subsequently filled with 1 mL/droplet expansion media (FIG. 9F). The floating droplet culture method was tested at 0, 17.5 revolutions per minute (RPM) and 35 RPM. A spinning speed of 17.5 RPM was chosen based on acceptable phenotypic stability and cellular expansion properties (FIGS. 6A-D). Fifty drops from the 17.5 RPM setting were cultured in each 8-day expansion period for lung scaffold recellularization experiments. After 8 days of culture, the Matrigel drops were digested with Dispase and the alveolar spheres were trypsinized to produce single cell suspensions prior to analysis and lung scaffold seeding.

Adherent Droplet Cell Culture

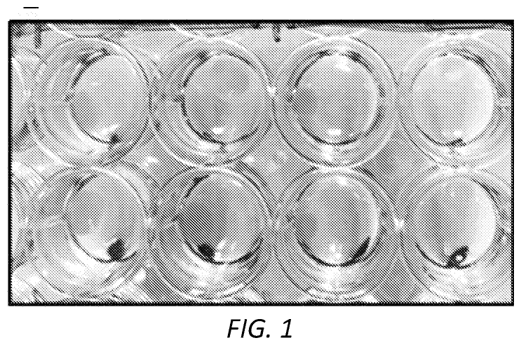

Matrigel based homogenous liquid precursor with suspended cells was aliquoted into 100 μL drops containing approximately 20,000 cells each. Drops were placed on tissue culture plastic in individual wells of a 12-well plate and allowed to gel at 37° C. for 20 minutes (FIG. 1). After stability of the gel drop was confirmed, 1 mL expansion media was added to each well and the plate was placed in a 37° C., 5% CO₂ incubator. Fifty drops were cultured in each 8-day expansion period. After 8 days of culture, the Matrigel drops were digested with Dispase (Corning, Corning, N.Y.) and the alveolar spheres were trypsinized to produce single cell suspensions prior to analysis and lung scaffold seeding.

Rat Lung Decellularization

Cadaveric rat lungs were decellularized as previously described (13). Briefly, rat lungs were explanted from outbred adult male Sprague-Dawley rats. The pulmonary artery (PA) was cannulated via the right ventricular outflow tract, followed by tracheal cannulation. The lungs were perfused with a 0.1% sodium dodecyl sulfate (SDS) (Fisher Scientific, Waltham, Mass.) solution via the PA cannula for 2 hours. The lung scaffold was then perfused with sterile deionized water for 15 minutes followed by perfusion with 1% Triton X-100 (Fisher Scientific, Waltham, Mass.), all via the PA cannula. Finally, the decellularized lung scaffolds were washed with a minimum of 3 L phosphate buffered saline (PBS) over the subsequent 48 hours prior to use.

Seeding Lungs for Culture

After left pneumonectomy, rat lung scaffolds were mounted in custom bioreactors prefilled 100 mL alveolar sphere expansion medium perfused for a minimum of 1 hour at a flow rate of 1 mL/min in a 37° C., 5% CO₂ incubator. Forty million iPSC-AECs were gravity seeded into the airway of each right lung with 50 mL expansion medium via the tracheal cannula. After seeding, the PA perfusion was paused for 90 minutes to allow for a static culture period promoting cell attachment to the scaffold. Perfusion was reinitiated at 1 mL/min for the next 16 hours, then increased to 3 mL/min for the remainder of the biomimetic culture period. Culture medium was changed every 48 hours for the 12-day culture period. Right upper and middle lobectomies were performed on post-seeding day 6. Tissue for RNA analysis was stored in Trizol (Fisher Scientific, Waltham, Mass.), and tissue for histologic analysis was fixed with 4% paraformaldehyde (PFA) (Westnet, Canton, Mass.) for 24 hours. On post-seeding day 12, the lower and accessory lobes were perfusion fixed via the tracheal cannula with 4% PFA for 24 hours.

Resazurin Assay

A resazurin cell metabolic assay was performed as previously described (7). Briefly, 80 mL spent media was mixed with PrestoBlue (Invitrogen, Waltham, Mass.) at a 1:20 dilution. Quadruplicate samples of the PrestoBlue mixture were saved in a 96-well flat bottom plate as controls. The mixture was then allowed to perfuse the biomimetic lung culture for 1 hour on experimental day 12. Upon completion, the spent media was sampled in quadruplicate and measured in a SpectraMax M3 multi-mode microplate reader (Molecular Devices, Sunnyvale, Calif.). The difference in fluorescence between the samples and controls was correlated to metabolic activity.

Histological Staining and Analysis

Alveolar spheres were embedded in Histogel (ThermoFisher, Waltham, Mass.) and paraffin-embedded prior sectioning. Fixed tissue sections were paraffin-embedded and sectioned. Tissue or cell sections mounted on glass slides were stained with hematoxylin and eosin for brightfield imaging. Tissue sections mounted on glass slides for immunofluorescent staining underwent antigen retrieval with a sodium citrate solution at high temperature and pressure and were permeabilized with 0.2% Triton X-100. Sections were then blocked with 10% fetal bovine serum (FBS) and 5% donkey serum (DS) (Sigma, St. Louis, Mo.). Primary antibodies were incubated overnight at 4° C. in a tris-buffered saline (TBS) with 0.5% DS solution then washed with TBS (1:50, Nkx2.1:ab72876, Abcam, Cambridge, UK; 1:200 SPC:ab3786, Abcam; 1:100 AQP5:ab92320, Abcam). Secondary antibodies were incubated for 2 hours at room temperature then washed with TBS (Alexa Fluor donkey anti-rabbit 594 or 647:ab150064 or ab150075, respectfully, Invitrogen). Slides were mounted with DAPI Fluoromount-G (Fisher Scientific, Waltham, Mass.). Images were captured using a Nikon Ti-PFS inverted microscope (Nikon, Tokyo, Japan). All fluorescent images for a given protein were captured with consistent exposure time and instrument gain. ImageJ software (National Institutes of Health, Bethesda, Md.) was used for analysis. Cell counts were obtained by isolating the DAPI color channel, subtracting the background signal, converting the image to a binary image, defining cell borders, and counting discrete nuclei. Image fluorescence was obtained by isolating the appropriate fluorescent channel, analyzing each image from a particular protein staining with consistent brightness and contrast, then calculating the mean fluorescence. Quantitative data was generated as a mean fluorescence per cell.

Spent Media Analysis from Biomimetic Lung Culture

The biomimetic lung culture media was changed every 48 hours and analyzed for pH, bicarbonate, lactate, and glucose concentration using an iSTAT (Abbott, Chicago, Ill.) point of care analyzer with CG4+cartridges (Abbott, Chicago, Ill.) and G cartridges (Abbott, Chicago, Ill.).

Relative Gene Expression Analysis

RNA was isolated by Trizol then reverse transcribed to cDNA by SuperScript Vilo Master Mix (Life Technologies, Carlsbad, Calif.). Gene expression was quantified by Taqman Assay with probes (see Key Resources Table for probe details) (Life Technologies, Carlsbad, Calif.) using the One Step Plus (Applied Biosystems, Foster City, Calif.) system. Gene expression was analyzed using the delta-delta method by normalizing to the housekeeping gene β-actin.

Flow Cytometric Analysis

Fluorescence activated cell sorting of Nkx2.1-GFP+/tdTomato+ cells was conducted using a FACSAria II (BD Biosciences, Franklin Lakes, N.J.). For phenotypic analysis, cells were fixed and permeabilized using BD Cytofix/Cytoperm (BD Biosciences, Franklin Lakes, N.J.) kit. Cells were stained with primary antibodies (1:250, SPC: ab40879, Abcam; 1:200 AQP5: ab92320, Abcam) for 30 minutes at 4° C., washed, then stained with secondary antibodies (1:200, Alexa Fluor donkey anti-rabbit 350 or 594: A10039 or ab150064, respectfully, Invitrogen) for 30 minutes at 4° C. Flow cytometric analysis was conducted using FlowJo software (BD Biosciences, Franklin Lakes, N.J.).

Statistical Methods

Data is presented as the mean±SEM. For tests of significance, a one-tailed Student's t-test with unequal variance was used to compare two populations. Significant was determined if p≤0.05. Data represented in FIGs. is assumed to be non-significant unless noted with an asterisk (*). For qPCR data, individual datapoints were excluded if they were determined to be greater than 3 standard deviations from the mean when including all datapoints in the descriptive statistic calculations. All statistical calculations were performed using Visual Basic for Applications.

### Example 1. Floating Culture Method

To enable a floating droplet culture, we designed an autoclavable silicon mold (FIGS. 9A, C-D). The rounded wells of the mold align with a multi-channel pipette allowing for rapid transfer of homogenous cell-laden gel-precursor droplets. The previously described manual droplet formation method, in which the gel must warm for 90 seconds in the pipette tip prior to use, was also performed (FIG. 1) (4). Following solidification, the cell-laden Matrigel droplets were transferred under sterile conditions to a flask for continued cellular expansion and culture (FIGS. 9E-F). A sterile spatula with a single cell laden MATRIGEL droplet with approximately 20,000 cells is shown in FIG. 9E. A flask with amagnetic stir rod containing cell laden MATRIGEL floating droplets in cell culture media is shown in FIG. 9F.

### Example 2. Quantification of iPSC-AEC Expansion

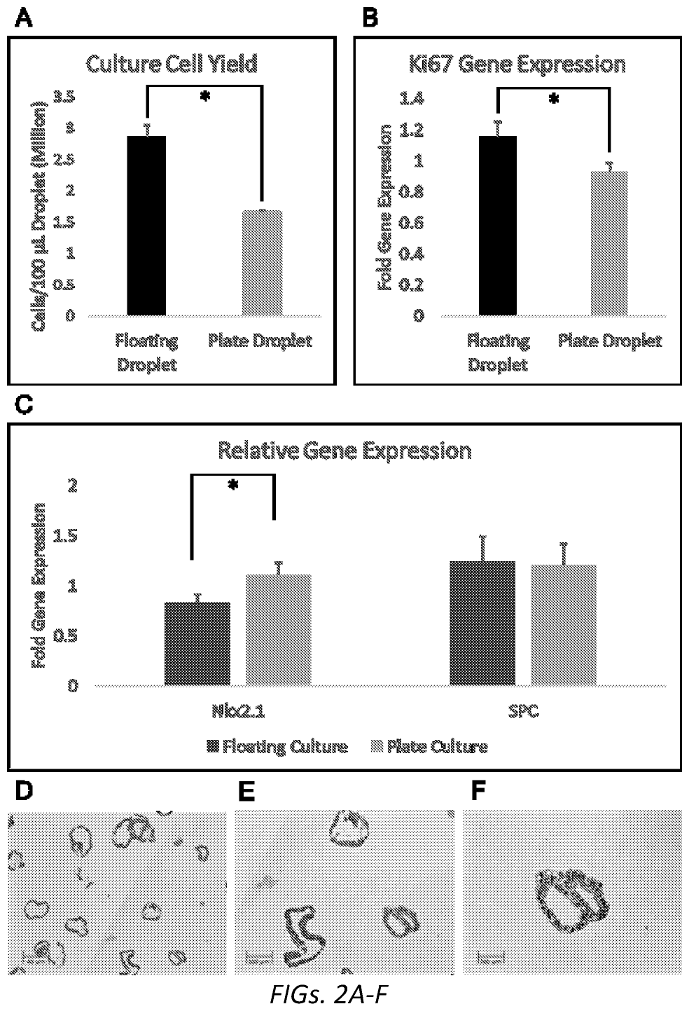

The floating droplet culture method produced significantly more cells during the 8-day period than the adherent droplet culture method (2.86 million (M) cells/droplet vs 1.66 M cells/droplet, respectively, p<0.01, FIG. 2A). Floating droplets cultured at a mechanical stirring rate of 17.5 RPM showed greater cellular expansion compared with higher and lower stirring speeds (FIG. 6A). Relative gene expression analysis showed a significantly increased expression of Ki-67 in the floating droplet culture cells compared to the adherent droplet culture cells (p=0.033) (FIG. 2B).

### Example 3. Characterization of Expanded iPSC-AECs

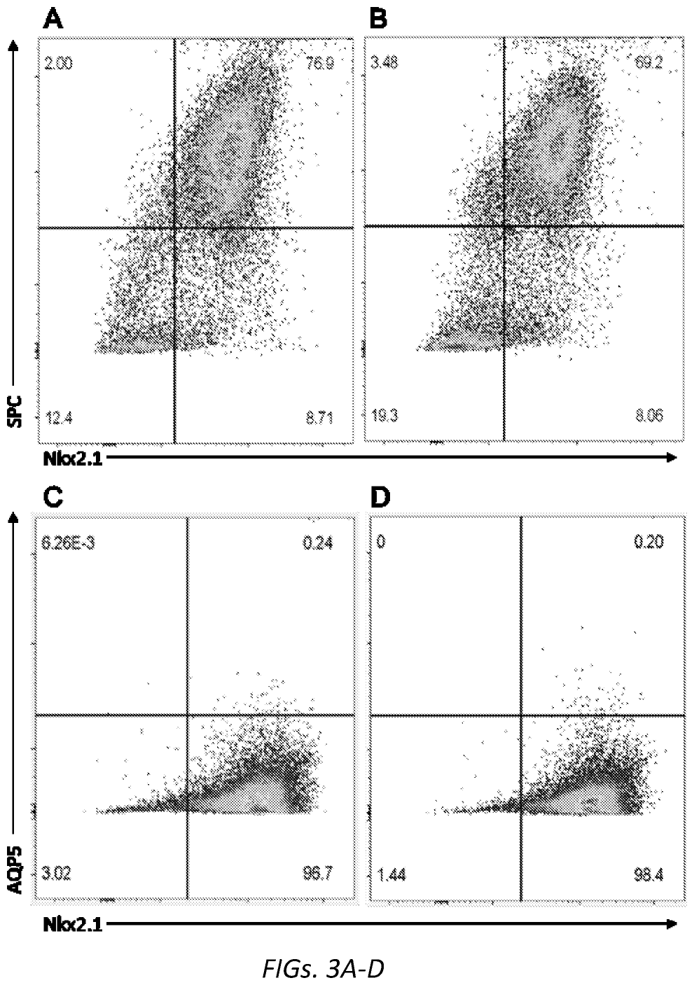

Quantitative polymerase chain reaction (PCR) analysis demonstrated comparable expression of SPC between the adherent and floating culture methods but showed lower gene expression for Nkx2.1 in the floating droplet culture (p=0.041) (FIG. 2C). Flow cytometric characterization demonstrated relatively preserved Nkx2.1 and SPC expression between the culture methods, but decreased Nkx2.1 expression in the floating culture method is noted (FIGS. 3A-B). This decrease in Nkx2.1 expression correlates with the significant difference noted in the PCR data for Nkx2.1 (FIG. 2C). In accordance with prior work, iPSC-AECs from the floating droplet method spontaneously formed alveolar spheres (4, 9) (FIGS. 2D-F). The type I AEC marker aquaporin 5 (AQP5) was analyzed with flow cytometry with no appreciable AQP5 expression from either culture condition (FIGS. 3C-D). When optimizing the floating droplet culture method mechanical stirring rate, the type II alveolar cell marker SPC was found to trend lower at high stirring speeds (FIGS. 6B-D).

### Example 4. Gene Expression after Biomimetic Lung Culture

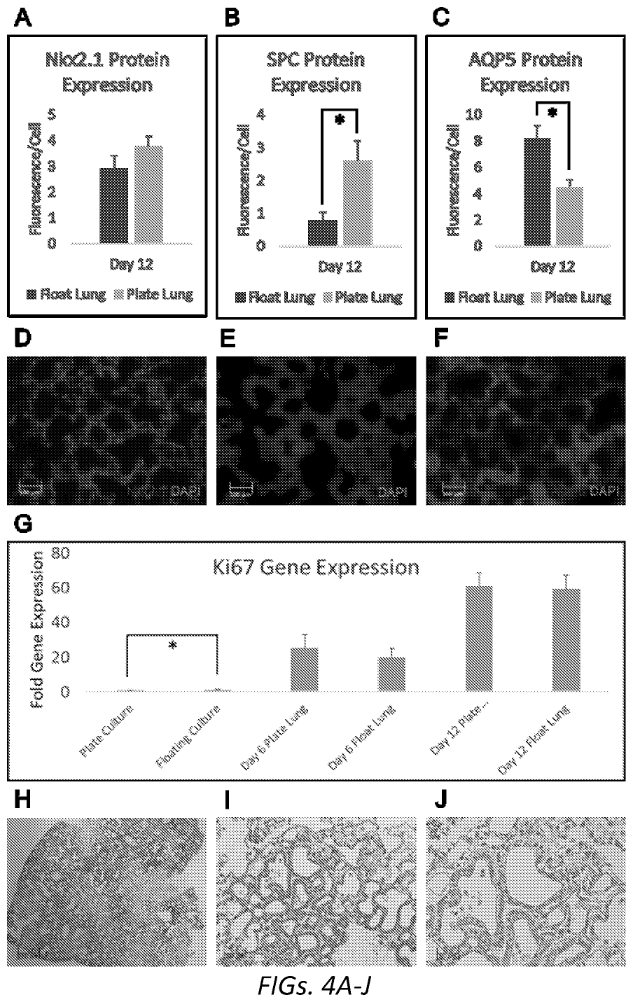

Immunohistochemical staining of the lungs following biomimetic culture revealed a trend toward higher expression of Nkx2.1 in lungs with the adherent droplet culture cells (p=0.059) which corresponds with the PCR data from the end of the cell expansion period (FIGS. 4A, D; FIG. 2C). Lungs with floating droplet culture cells showed a significantly decreased SPC expression (p<0.01) and a significantly increased AQP5 expression (p<0.001) (FIGS. 4B-C, E-F). Quantitative PCR demonstrated a consistent increase in Ki67 expression from the culture period through the end of the biomimetic lung culture with similar expression between each group (FIG. 4G).

### Example 5. Cellular Metabolism During Biomimetic Lung Culture

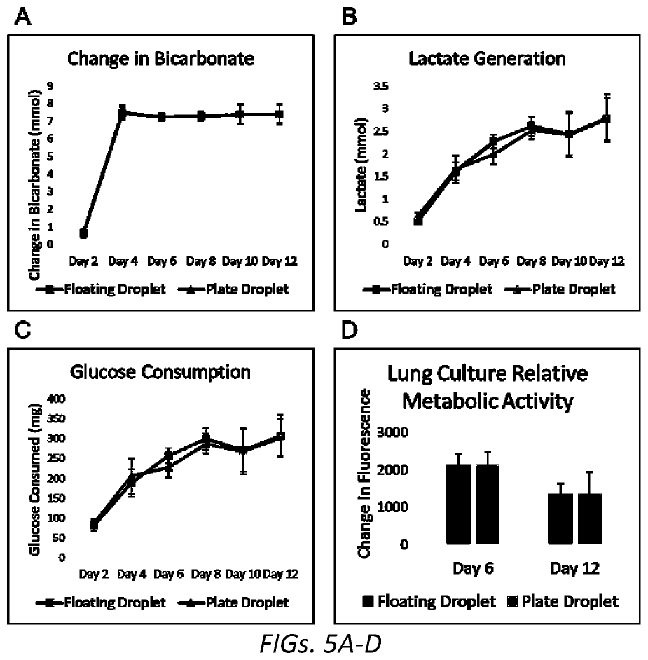

Spent media from the biomimetic lung culture demonstrated a comparable trend in bicarbonate change, lactate generation, and glucose consumption from both groups throughout the culture period (FIGS. 5A-C). The trends support data that proliferation continues throughout the culture period (FIG. 4G). The resazurin cell viability assay on culture days 6 and 12 showed similar mitochondrial conversion of resazurin to resorufin, as detected by a fluorescent plate reader, indicating similar cellular energy consumption between both groups (FIG. 5D).

### Example 6. Generating AEC from Additional iPSC Lines

To test if our scalable culture protocol is applicable for other human induced pluripotent stem cells derived alveolar epithelial cells (iPSC-AECs), we generate AECs from two other human iPSC lines. iPSC-17 cell line carries Nkx2.1-GFP and Surfactant Protein (SPC)-TdTomato reporters, while SPC2 cell line carried only SPC-TdTomato reporter. After 4 weeks of step-wised differentiation and 2 times of flow cytometry sorting, we purify SPC-TdTomato expressing AECs from iPSC-17 and SPC2 cell lines separately. 100% Matrigel is mixed with AECs then aliquoted into 100 μL drops containing approximately 20,000 cells each for the subsequent droplet or plate culture with 1 ml medium per drop, while medium is changed every other day.

Cells cultured for 8 days are harvested and counted for cell yield comparison between different methods, then fixed for flow cytometry analysis of AT2 cell marker SPC, AT1 cell marker AQP5, and lung epithelial progenitor marker Nkx2.1. Harvested cells are used for realtime PCR analysis and H&E staining of AEC marker genes. Data is generated from another two iPSC-17-AEC and SPC2-AECs to prove that our scalable method could promote proliferation while keeping AT2 phenotype in different iPSC-AEC cell lines.

### REFERENCES

(1) “Report of the U.S. Organ Procurement and Transplantation Network and the Scientific Registry of Transplant Recipients: Transplant Data,” optn.transplant.hrsa.gov/data/view-data-reports/national-data/.
(2) Egan, T. M., and Edwards, L. B. (2016). J Heart Lung Transplant, vol. 35, no. 4, pp. 433-9, April, 2016.10.1016/j.healun.2016.01.010
(3) Adegunsoye, A., Strek, M. E., Garrity, E., Guzy, R., and Bag, R. (2017). Chest, vol. 152, no. 1, pp. 150-164, July, 2017.10.1016/j.chest.2016.10.001
(4) Hawkins, F., Kramer, P., Jacob, A., Driver, I., Thomas, D. C., McCauley, K. B., Skvir, N., Crane, A. M., Kurmann, A. A., Hollenberg, A. N. et al. (2017). J Clin Invest, vol. 127, no. 6, pp. 2277-2294, Jun. 1, 2017.10.1172/JCI89950
(5) Jacob, A., Morley, M., Hawkins, F., McCauley, K. B., Jean, J. C., Heins, H., Na, C. L., Weaver, T. E., Vedaie, M., Hurley, K. et al. (2017). Cell Stem Cell, vol. 21, no. 4, pp. 472-488 e10, Oct. 5, 2017.10.1016/j.stem.2017.08.014
(6) McCauley, K. B., Hawkins, F., Serra, M., Thomas, D. C., Jacob, A., and Kotton, D. N. (2017). Cell Stem Cell, vol. 20, no. 6, pp. 844-857 e6, Jun. 1, 2017.10.1016/j.stem.2017.03.001
(7) Guillot, L., Nathan, N., Tabary, O., Thouvenin, G., Le Rouzic, P., Corvol, H., Amselem, S., and Clement, A. (2013). Int J Biochem Cell Biol, vol. 45, no. 11, pp. 2568-73, November, 2013.10.1016/j.biocel.2013.08.009
(8) Gilpin, S. E., Charest, J. M., Ren, X., Tapias, L. F., Wu, T., Evangelista-Leite, D., Mathisen, D. J., and Ott, H. C. (2016). Biomaterials, vol. 108, pp. 111-9, November, 2016.10.1016/j.biomaterials.2016.08.055
(9) Wu, T. G. D. Z., L.; Ohata, K.; Gilpin, S.; Ott, H., Engineering Distal Pulmonary Epithelium from Induced Pluripotent Stem Cells (iPSCs) Derived Alveolar Cells., Poster, American Association for Thoracic Surgery 99th Annual Meeting, 2019.
(10) Mercer, R. R., Russell, M. L., Roggli, V. L., and Crapo, J. D. (1994). Am J Respir Cell Mol Biol, vol. 10, no. 6, pp. 613-24, June, 1994.10.1165/ajrcmb.10.6.8003339
(11) Kurmann, A. A., Serra, M., Hawkins, F., Rankin, S. A., Mori, M., Astapova, I., Ullas, S., Lin, S., Bilodeau, M., Rossant, J. et al. (2015). Cell Stem Cell, vol. 17, no. 5, pp. 527-42, Nov. 5, 2015.10.1016/j.stem.2015.09.004
(12) Huang, S. X., Islam, M. N., O'Neill, J., Hu, Z., Yang, Y. G., Chen, Y. W., Mumau, M., Green, M. D., Vunjak-Novakovic, G., Bhattacharya, J. et al. (2014). Nat Biotechnol, vol. 32, no. 1, pp. 84-91, January, 2014.10.1038/nbt.2754
(13) Ott, H. C., Clippinger, B., Conrad, C., Schuetz, C., Pomerantseva, I., Ikonomou, L., Kotton, D., and Vacanti, J. P. (2010). Nat Med, vol. 16, no. 8, pp. 927-33, August, 2010.10.1038/nm.2193
(14) Caliari, S. R., and Burdick, J. A. (2016). Nat Methods, vol. 13, no. 5, pp. 405-14, Apr. 28, 2016.10.1038/nmeth.3839
(15) Yamamoto, Y., Gotoh, S., Korogi, Y., Seki, M., Konishi, S., Ikeo, S., Sone, N., Nagasaki, T., Matsumoto, H., Muro, S. et al. (2017). Nat Methods, vol. 14, no. 11, pp. 1097-1106, November, 2017.10.1038/nmeth.4448
(16) Gu, Q., Tian, H., Zhang, K., Chen, D., Chen, D., Wang, X., and Zhao, J. (2018). Cell Physiol Biochem, vol. 48, no. 1, pp. 215-226, 2018.10.1159/000491721
(17) Li, Y. Q. (2018). Curr Stem Cell Res Ther, vol. 13, no. 8, pp. 676-681, 2018.10.2174/1574888X13666180723105028

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of generating an expanded population of alveolar epithelial cells (AECs), the method comprising:
(a) providing a first population of AECs;
(b) mixing the first population of AECs into a hydrogel precursor;
(c) allowing or promoting gelation of the hydrogel precursor to form a droplet; and
(d) culturing the droplets in suspension in moving media sufficient for expansion of the first population, thereby generating an expanded population of AECs.

2. The method of claim 1, wherein the first population of AECs comprises induced pluripotent stem cell (iPSC)-derived AECs.

3. The method of claim 2, wherein the iPSC-derived AECs are obtained by a method comprising:
providing an initial population of iPSC;
culturing the iPSC under conditions sufficient for definitive endodermal differentiation, then under conditions sufficient for anteriorized endodermal differentiation, and then under conditions sufficient for ventralized endodermal differentiation, thereby obtaining a population of iPSC-derived AECs.

4. The method of claim 1, wherein the droplet has a maximal diameter of 2-10 mm.

5. The method of claim 1, wherein the hydrogel is a natural or synthetic hydrogel scaffold.

6. The method of claim 5, wherein the natural hydrogel scaffold comprises extracellular matrix (ECM), collagen, fibrin, bone sialoprotein, vitronectin, alginate, or laminin.

7. The method of claim 5, wherein the synthetic hydrogel scaffold comprises a synthetic polymeric scaffold selected from poly(2-(methacryloyloxy) ethyl dimethyl-(3-sulfopropyl)ammonium hydroxide) (PMEDSAH), polyacrylamide (PAM), poly(sodium 4-stryenesulfonate) (PSS), poly(methyl vinylether-alt-maleic anhydride), and poly(ethylene glycol) (PEG) hydrogels.

8. The method of claim 1, wherein allowing or promoting gelation of the hydrogel comprises providing a temperature, chemical, or light sufficient to initiate crosslinking of the hydrogel scaffold.

9. The method of claim 1, wherein the moving media is spinning or flowing culture.

10. The method of claim 1, wherein the expanded population of AECs comprises cells that express Nkx2.1 and aquaporin 5 (AQP5) or Surfactant Protein C (SPC).

11. An expanded population of AECs produced by the method of claim 1.

12. A method of providing a bioartificial lung organ, the method comprising:
providing the expanded population of AECs of claim 11;
providing a (cell-free) lung tissue matrix including an airway and vasculature;
seeding the lung tissue matrix with the expanded population of AECs through the airway, with endothelial cells through the vasculature, and with mesenchymal cells through either one or both of the airway and the vasculature; and
maintaining the matrix under conditions sufficient for the formation of a functional epithelium in the airways and functional vasculature.

13. A mold apparatus, comprising:
a mold body comprising a flexible polymeric material, the mold body defining a first cavity and a second cavity, the first and second cavities each having a radius of between 0.5 mm and 5 mm and configured to receive a composition,
the mold body further defining a first channel that extends along a longitudinal axis that intersects the first and second cavities, wherein the first channel is defined by a depth dimension configured to limit a volume amount of the composition in the first and second cavities to a maximum volume amount of about 50 μL to about 150 μL.

14. (canceled)

15. (canceled)

16. (canceled)

17. (canceled)

18. (canceled)

19. The mold apparatus of claim 13, wherein each cavity is defined by a hemispherically shaped surface, or is configured to form spherically shape compositions or hemi-spherically shaped compositions.

20. (canceled)

21. (canceled)

22. (canceled)

23. A method of forming shaped gel compositions, the method comprising adding a composition to cavities of the mold apparatus of claim 13, the composition being a liquid comprising a biologic;
forming a plurality of semi-solid or solid compositions in the cavities of the mold; and
removing the semi-solid or solid compositions from the cavities of the mold.

24. The method of claim 23, wherein the liquid is a hydrogel precursor and the biologic comprises cells.

25. (canceled)

26. (canceled)

27. (canceled)

28. (canceled)

29. (canceled)

30. The method of claim 23, wherein the semi-solid or solid compositions are spherical or semi-spherical.

31. The method of claim 1, further comprising:
after step (b), transferring the mixture to a mold apparatus to form spherical or semi-spherical droplets, and then after gelation of the hydrogel precursor in step (c), removing the droplets from the mold apparatus.

32. An expanded population of AECs produced by the method of claim 31.

33. A method of providing a bioartificial lung organ, the method comprising:
providing the expanded population of AECs of claim 32;
providing a (cell-free) lung tissue matrix including an airway and vasculature;
seeding the lung tissue matrix with the expanded population of AECs through the airway, with endothelial cells through the vasculature, and with mesenchymal cells through either one or both of the airway and the vasculature; and
maintaining the matrix under conditions sufficient for the formation of a functional epithelium in the airways and functional vasculature.
